(19) United States
(12) Patent Application Publication
(10) Pub. No.: US 2012/0171219 A1
(43) Pub. Date: Jul. 5, 2012

(54) USE OF PKC-ZETA AS A BREAST CANCER TUMORIGENIC BIOMARKER AS WELL AS A TARGET FOR TREATMENT OF BREAST CANCER

(75) Inventors: Mildred Acevedo-Duncan, Plant City, FL (US); Diondra D. Hill, Tampa, FL (US)

(73) Assignees: United States Department of Veterans Affairs, Washington, DC (US); University of South Florida, Tampa, FL (US)

(21) Appl. No.: 13/309,018

(22) Filed: Dec. 1, 2011

Related U.S. Application Data
(60) Provisional application No. 61/418,585, filed on Dec. 1, 2010.

(51) Int. Cl. A61K 39/395; G01N 33/574; G01N 21/76; A61P 35/00; G01N 21/64; C12Q 1/48; A61K 31/7088

(52) U.S. Cl. 424/158.1; 435/15; 435/7.4; 514/44 A

(57) ABSTRACT

The present invention provides use of protein kinase C-zeta (PKC-ζ) as a diagnostic biomarker for breast cancer tumorigenesis. Also provided are uses of PKC-zeta inhibitors for inhibiting breast cancer tumorigenesis and for treatment of breast cancer.

### CROSS-REFERENCE TO RELATED APPLICATION

This application claims the benefit of U.S. Provisional Application Ser. No. 61/418,585, filed Dec. 1, 2010, which is hereby incorporated by reference in its entirety.

### FIELD OF INVENTION

The present invention provides assays for prediction and detection of breast cancer tumorigenesis, as well as methods for treatment of breast cancer.

### BACKGROUND

Breast cancer is the most common female malignancy and the leading cause of cancer-related death among women. The 2011 cancer statistics estimated that 230,000 new cases of invasive breast cancer will be diagnosed that year and would result in 40,000 new deaths. In North America, breast cancer accounts for about 27% of all female cancers and 15%-20% of all female cancer mortalities. Also, approximately 1,700 men will be diagnosed with breast cancer and 450 will die each year. Despite significant educational efforts, improved diagnostic techniques, and rigorous therapies, breast cancer control remains static.

Protein kinase C (PKC) is a family of fourteen known isozymes which are found in varying ratios in the cytoplasmic and membrane fraction of cells depending on the type of tissue and its physiological state. PKC isozymes can be classified into three groups. Group I includes Ca²⁺ dependent isozymes: cPKC-α, cPKC-β_{I}, cPKC-β_{II}, and cPKC-y. Isozymes in group II (nPKC-ε, nPKC-δ, nPKC-η and PKC-θ) are Ca²⁺ independent. Group III includes the atypical PKCs (aPKC-ι, aPKC-ζ, PKMζ (a brain-specific isoform of PKC-zeta generated from an alternative transcript), aPKC-μ(protein kinase D) and aPKC-ν), which are insensitive to both diacylglycerol and calcium and neither bind to nor are activated by phorbol esters.

PKC regulates cellular functions, metabolism, and proliferation by phosphorylating proteins in response to transmembrane signals from hormones, growth factors, neuro-transmitters, and pharmacological agents. Activation of PKC by various agonists (including radiation) results in altered transcription of a considerable number of genes. Some PKC isozymes are transiently translocated from the cytosol to a membrane structure. Membrane association leads to binding alterations in PKC's regulatory subunit (phospholipid-/diacylglycerol/phorbol ester) and its 50 KD catalytic domain (ATP/substrate). For PKCs to be activated, phosphoinositide-dependent kinase (PDK-1) docks on the carboxyl terminus of unphosphorylated PKC, PDK-1 phosphorylates PKCs on the activation loop, and upon release of PDK-1, the carboxyl terminus is unmasked and allows autophosphorylation. This sequence of phosphorylation events is required before PKCs are able to respond to cofactor second messengers (phosphatidylserine/diacylglycerol). Proteolytic degradation of membrane PKC leads to its down-regulation. PKC is the major receptor for tumor promoting phorbol esters, but the extent of PKC involvement in cellular malignancy is not clearly defined.

### BRIEF SUMMARY

The present invention provides use of protein kinase C-zeta (PKC-ζ) as a biomarker for prediction and/or detection of breast cancer tumorigenesis, as well as a therapeutic target for breast cancer therapy.

One aspect of the present invention pertains to the use of PKC-zeta as a biomarker for prediction and/or detection of breast tumorigenesis and/or breast cancer.

In one embodiment, the present invention provides a method for predicting whether a subject is at risk of breast tumorigenesis and/or developing breast cancer, comprising:

(a) obtaining a biological sample from a subject;

(b) detecting in the sample a level of expression for PKC-zeta; and

(c) comparing the expression level in (b) to a level of expression in a normal control, wherein overexpression of PKC-zeta, with respect to the control, indicates that the subject is at risk of breast tumorigenesis and/or developing breast cancer.

In another embodiment, the present invention provides a method for predicting the risk of recurrence of breast cancer in a subject, wherein the subject had received treatment for breast cancer and, as a result of the treatment, does not have detectable breast cancer cells, wherein the method comprises:

(a) obtaining a biological sample from a subject who has received treatment for breast cancer;

(b) detecting in the sample a level of expression for PKC-zeta; and

(c) comparing the expression level in (b) to a level of expression in a normal control, whereby overexpression of PKC-zeta with respect to the control indicates that the subject is at risk of breast cancer recurrence.

In a preferred embodiment, the biological sample is obtained from the breast(s) of the subject. In certain embodiments, the biological samples obtained from the breast(s) include, but are not limited to, samples containing breast tissue, breast cells, and fluid (e.g., interstitial fluid) of the breast(s). In certain embodiments, the biological sample is a biopsy sample obtained from a cyst, tumor, polyp, lump, and/or other tissues, cells, or fluid of the breast(s).

In another aspect, the present invention provides methods for inhibiting breast tumorigenesis and/or for treatment of breast cancer. In one embodiment, the method comprises administering, to a subject in need of such treatment, an effective amount of a PKC-zeta inhibitor. In one embodiment, the present invention administers agents that specifically inhibit PKC-zeta but do not substantially inhibit other PKC isoforms.

PKC-zeta inhibitors useful according to the present invention include, but are not limited to, agents that inhibit PKC-zeta activity; and agents that reduce or inhibit the expression of PKC-zeta, such as agents that inhibit the transcription, translation, and/or processing of PKC-zeta.

### BRIEF DESCRIPTION OF THE SEQUENCES SEQ ID NO: 1 is an amino acid sequence of human protein kinase C-zeta type 1 isoform (PKC-ζ) (GenBank Accession No. AAA36488).

### BRIEF DESCRIPTION OF THE DRAWING

FIG. 1 shows that PKC-zeta, which is not expressed in normal and benign breast tissues, is significantly overexpressed in malignant breast tumor. The amount of PKC-zeta in breast tissues is determined by Western Blotting.

### DETAILED DESCRIPTION

The present invention provides use of protein kinase C-zeta (PKC-ζ) as a biomarker for prediction and/or detection of breast tumorigenesis or breast cancer, as well as a therapeutic target for breast cancer therapy. The invention is based on the discovery that PKC-zeta is not expressed, or is minimally expressed, in normal and benign breast tissues, but is significantly overexpressed in malignant breast tumors. The increase in PKC-ζ in malignant breast cancer biopsies is greater than 18,000 fold when compared to that of normal and benign breast tissue.

### DEFINITIONS

To facilitate the understanding of the subject matter disclosed herein, a number of terms, abbreviations or other shorthand as used herein are defined below. Any term, abbreviation or shorthand not defined is understood to have the ordinary meaning used by a skilled artisan contemporaneous with the submission of this application.

The term “subject,” as used herein, describes an mammal including human and non-human mammals including, but not limited to, apes, chimpanzees, orangutans, monkeys, dogs, cats, horses, pigs, sheep, goats, mice, rats, and guinea pigs.

The term “tumorigenesis, as used herein, refers to its ordinary meaning that is the development of a malignant tumor.

### Prediction and Diagnosis of Breast Tumorigenesis and Breast Cancer

One aspect of the present invention pertains to the use of PKC-zeta as a biomarker for prediction and/or detection of breast tumorigenesis and/or breast cancer.

In one embodiment, the present invention provides a method for predicting whether a subject is at risk of breast tumorigenesis and/or developing breast cancer, comprising:

(a) obtaining a biological sample from a subject;

(b) detecting in the sample a level of expression for PKC-zeta; and

(c) comparing the expression level in (b) to a level of expression in a normal control, wherein overexpression of PKC-zeta, with respect to the control, indicates that the subject is at risk of breast tumorigenesis and/or developing breast cancer.

In another embodiment, the present invention provides a method for diagnosing the presence of breast tumorigenesis or breast cancer in a subject, comprising:

(a) obtaining a biological sample from a subject;

(b) detecting in the sample a level of expression for PKC-zeta; and

(c) comparing the expression level in (b) to a level of expression in a normal control, wherein overexpression of PKC-zeta, with respect to the control, indicates the presence of breast tumorigenesis or breast cancer in the subject.

In a further embodiment, the degree of PKC-zeta overexpression, when compared to a normal control, corresponds to the tumorigenic and/or proliferative potential of the breast tumor/tumor cells of the subject, wherein a higher degree of PKC-zeta overexpression in the subject's sample indicates a higher tumorigenic and/or proliferative potential of the breast tumor/tumor cells of the subject.

In a further embodiment, the degree of PKC-zeta overexpression, when compared to a normal control, corresponds to the rate of proliferation of the breast cancer or cancer cells, wherein a higher degree of PKC-zeta overexpression in the subject's sample indicates a higher rate of proliferation of the breast cancer or cancer cells in the subject.

In another embodiment, the present invention provides a method for predicting the risk of recurrence of breast cancer in a subject, wherein the subject has received treatment for breast cancer and, as a result of the treatment, does not have detectable breast cancer cells, wherein the method comprises:

(a) obtaining a biological sample from a subject who has received treatment for breast cancer;

(b) detecting in the sample a level of expression for PKC-zeta; and

(c) comparing the expression level in (b) to a level of expression in a normal control, whereby overexpression of PKC-zeta with respect to the control indicates that the subject is at risk of breast cancer recurrence.

In a preferred embodiment, the biological sample is obtained from the breast(s) of the subject. In certain embodiments, the biological samples obtained from the breast(s) include, but are not limited to, samples containing breast tissue, breast cells, and fluid (e.g., interstitial fluid) of the breast(s). In certain embodiments, the biological sample is a biopsy sample obtained from a cyst, tumor, polyp, lump, and/or other tissues, cells, or fluid of the breast(s). In other embodiments, the biological sample is a blood (such as whole blood, plasma, and serum) sample or urine sample.

In one embodiment, the biological sample is obtained from a tumor, polyp, or cyst of a subject's breast, wherein overexpression of PKC-zeta in the subject's sample, when compared to the control, indicates the tumor, polyp, or cyst is a malignant tumor and/or will become malignant.

In one embodiment, the control level of PKC-zeta expression is determined by measuring PKC-zeta expression in a healthy population that do not have breast cancer and/or breast tumorigenesis. In another embodiment, the control level of PKC-zeta expression is determined by measuring PKC-zeta expression in a control population that have benign breast tumors but do not have breast cancer and/or breast tumorigenesis.

The level of PKC-zeta expression can be determined based on mRNA levels or protein levels. Determination of PKC-zeta expression can be made qualitatively, semi-quantitatively, or quantitatively. Sequences of PKC-zeta proteins and mRNAs of a variety of mammalian species are publicly available and can be obtained from, for example, the GenBank database. For instance, human PKC-zeta protein has an amino acid sequence of SEQ ID NO:1 (GenBank Accession No. AAA36488). One of ordinary skill in the art, having the benefit of the present disclosures, can easily use PKC-zeta sequences of a mammalian species of interest to practice the present invention.

Methods for determining PKC-zeta expression level are well known in the art, including but not limited to, Western blots, Northern blots, Southern blots, enzyme-linked immunosorbent assay (ELISA), immunoprecipitation, immunofluorescence, radioimmunoassay, flow cytometry, immunocytochemistry, nucleic acid hybridization techniques, nucleic acid reverse transcription methods, nucleic acid amplification methods, and any combination thereof.

In one embodiment, the level of PKC-zeta protein expression is determined by contacting the biological sample with an antibody that specifically recognizes, or specifically binds to, PKC-zeta protein; and detecting the complex between the antibody and the PKC-zeta protein. In preferred embodiments, the PKC-zeta-specific antibody does not recognize or bind to any PKC isozyme that is not PKC-zeta. In certain embodiments, the level of PKC-zeta expression can be determined by immunoassays including, but not limited to, radioimmunoassay, Western blot assay, ELISA, immunofluorescent assay, enzyme immunoassay, immunoprecipitation, chemiluminescent assay, immunohistochemical assay, dot blot assay, and slot blot assay.

A contacting step in the assay (method) of the invention can involve contacting, combining, or mixing the biological sample and the solid support, such as a reaction vessel, microvessel, tube, microtube, well, multi-well plate, or other solid support.

Samples and/or PKC-zeta-specific binding agents may be arrayed on the solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or “physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or 1536-well plates (or trays). Optionally, PKC-zeta-specific binding agents may be immobilized on the solid support.

In a further embodiment, the diagnostic assay of the present invention is used in combination with other routine breast cancer diagnostic or screening techniques, such as X rays (e.g., mammography), ultrasound, magnetic resonance imaging (MRI), needle biopsies, stereotactic breast biopsies, MRI-guided breast biopsies, and surgical biopsies.

In another aspect, the present invention includes kits comprising the required elements for detecting PKC-zeta. Preferably, the kits comprise a container for collecting a sample, such as breast tissue or fluid sample from a patient, and an agent for detecting the presence of PKC-zeta in the sample. The agent may be any binding agent specific for PKC-zeta, such as but not limited to antibodies and aptamers. The components of the kits can be packaged either in aqueous medium or in lyophilized form.

The methods of the invention can be carried out using a diagnostic kit for qualitatively or quantitatively detecting PKC-zeta in a sample. By way of example, the kit can contain binding agents specific for PKC-zeta, for example, antibodies against the antibodies labeled with an enzyme; and a substrate for the enzyme. The kit can also contain a solid support such as microtiter multi-well plates, standards, assay diluent, wash buffer, adhesive plate covers, and/or instructions for carrying out a method of the invention using the kit.

As indicated above, kits of the invention include reagents for use in the methods described herein, in one or more containers. The kits may include specific internal controls, and/or probes, buffers, and/or excipients, separately or in combination. Each reagent can be supplied in a solid form or liquid buffer that is suitable for inventory storage. Kits may also include means for obtaining a sample from a host organism or an environmental sample.

Kits of the invention can be provided in suitable packaging. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like. Preferably, the solid matrix is a structure having a surface that can be derivatized to anchor an oligonucleotide probe, primer, molecular beacon, specific internal control, etc. Preferably, the solid matrix is a planar material such as the side of a microtiter well or the side of a dipstick. In certain embodiments, the kit includes a microtiter tray with two or more wells and with reagents including primers, probes, specific internal controls, and/or molecular beacons in the wells.

Kits of the invention may optionally include a set of instructions in printed or electronic (e.g., magnetic or optical disk) form, relating information regarding the components of the kits and/or how to make various determinations (e.g., PKC-zeta levels, comparison to control standards, etc.). The kit may also be commercialized as part of a larger package that includes instrumentation for measuring other biochemical components.

### Treatment of Breast Cancer and Inhibition of Breast Tumorigenesis

In another aspect, the present invention provides methods for inhibiting breast tumorigenesis and/or for treatment of breast cancer. In one embodiment, the method comprises administering, to a subject in need of such treatment, an effective amount of a PKC-zeta inhibitor. In one embodiment, the present invention administers agents that specifically inhibit PKC-zeta but do not substantially inhibit other PKC isoforms or isozymes.

The term “treatment” or any grammatical variation thereof (e.g., treat, treating, and treatment etc.), as used herein, includes but is not limited to, ameliorating or alleviating a symptom of a disease or condition; reducing or delaying recurrence of a condition; reducing, suppressing, inhibiting, lessening, or affecting the progression and/or severity of an undesired physiological change or a diseased condition. For instance, treatment includes, for example, preventing, inhibiting, or slowing the rate of formation of a malignant breast tumor or development of a benign breast tumor into malignant; slowing the growth and/or proliferation of breast cancer cells; and reducing the size of malignant breast tumor.

The term “effective amount,” as used herein, refers to an amount that is capable of treating or ameliorating a disease or condition or otherwise is capable of producing an intended therapeutic effect. In certain embodiments, the effective amount enables a 5%, 10%, 20%, 30%, 40%, 50%, 75%, 90%, 95%, 99% or 100% reduction in the rate of formation of a malignant breast tumor. In certain embodiments, the effective amount enables a 5%, 10%, 15%, 20%, 25%, 30%, 35% or 40% reduction in the size of malignant breast tumor.

In an embodiment, a subject in need of the treatment of the present invention has, or is diagnosed of having, a malignant breast tumor or breast cancer. In another embodiment, a subject in need of the treatment of the present invention is at risk of having breast tumorigenesis. In another embodiment, a subject in need of the treatment of the present invention has, or is diagnosed of having, tumorigenic breast cells; however, no malignant breast tumor has formed yet. In another embodiment, a subject in need of the treatment of the present invention has, or is diagnosed of having, pre-malignant breast tumor cells, pre-cancerous breast cells, and/or cancer stem cells. In another embodiment, a subject in need of the treatment of the present invention has PKC-zeta overexpression in the breast(s). In an embodiment, the PCK-zeta-specific inhibitor is delivered to the breast tissue in need of such treatment.

In an embodiment, the present invention provides a method for treating breast cancer and/or inhibiting breast tumorigenesis. In an embodiment, the present invention can be used to treat or ameliorate primary breast cancer, in which cancer cells originated from breast tissue have not spread past the breast to distant parts of the body. In another embodiment, the present invention can be used to treat metastatic breast cancer. In a specific embodiment, the present invention can be used to treat or ameliorate non-invasive and/or invasive breast cancer. In another embodiment, the present invention can be used to inhibit or prevent the formation of a malignant breast tumor.

In certain embodiments, the present invention can be used to treat or ameliorate breast cancer, including ductal carcinoma in-situ (DCIS), invasive ductal carcinoma (IDC), lobular carcinoma in-situ (LCIS), invasive lobular carcinoma (LCIS), medullary carcinoma, malignant phyllode tumor, tubular carcinoma, mucinous carcinoma, metastatic adenocarcinoma, and inflammatory breast cancer.

In an embodiment, the present invention excludes the administration of PKC inhibitors that also inhibit the expression and/or activity of a PKC isoform or isozyme that is not PKC-zeta including, but not limited to, antibodies, binding partners, and/or aptamers that bind to a PKC protein isoform or isozyme that is not PKC-zeta; antisense nucleic acid molecules that inhibit the expression of a PKC protein isoform that is not PKC-zeta; and compounds (such as chelerythrine chloride) that inhibit a PKC protein isoform that is not PKC-zeta.

### PKC-Zeta Inhibitors

The present invention pertains to uses of PKC-zeta inhibitors for preventing and/or inhibiting breast tumorigenesis and for treatment of breast cancer. PKC-zeta inhibitors useful according to the present invention include, but are not limited to, agents that inhibit PKC-zeta activity; and agents that reduce or inhibit the expression of PKC-zeta, such as agents that inhibit the transcription, translation, and/or processing of PKC-zeta.

Agents that inhibit PKC-zeta activity include, but are not limited to, anti-PKC-zeta antibodies, aptamers, PKC-zeta binding partners, and small molecule inhibitors of PKC-zeta.

In one embodiment, the PKC-zeta inhibitor is an antibody that binds specifically to PKC-zeta. In a further specific embodiment, the PKC-zeta inhibitor is an antibody that binds specifically to human PKC-zeta. In some embodiments, PKC-zeta inhibitors include PKC-zeta antibodies that bind specifically to PKC-zeta proteins of non-human animals including, but not limited to, apes, chimpanzees, orangutans, monkeys, dogs, cats, horses, pigs, sheep, goats, mice, rats, and guinea pigs. The skilled artisan could easily construct PKC-zeta-specific antibodies to specifically target any PKC-zeta proteins publicly known. In a specific embodiment, the PKC-zeta inhibitor is an antibody or aptamer that binds specifically to a human PKC-zeta of SEQ ID NO:1.

“Specific binding” or “specificity” refers to the ability of a protein to detectably bind an epitope presented on a protein or polypeptide molecule of interest, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific target molecule versus nonspecific binding to other irrelevant molecules.

Anti-PKC-zeta antibodies of the present invention can be in any of a variety of forms, including intact immunoglobulin molecules, fragments of immunoglobulin molecules such as Fv, Fab and similar fragments; multimers of immunoglobulin molecules (e.g., diabodies, triabodies, and bi-specific and tri-specific antibodies, as are known in the art; see, e.g., Hudson and Kortt, J. Immunol. Methods 231:177 189, 1999); fusion constructs containing an antibody or antibody fragment; and human or humanized immunoglobulin molecules or fragments thereof.

Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Antibodies of the present invention include polyclonal and monoclonal antibodies. The term “monoclonal antibody,” as used herein, refers to an antibody or antibody fragment obtained from a substantially homogeneous population of antibodies or antibody fragments (i.e. the individual antibodies within the population are identical except for possible naturally occurring mutations that may be present in a small subset of the antibody molecules).

A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature, 1975, 256:495-497, the disclosure of which is herein incorporated by reference. An exemplary hybridoma technology is described by Niman et al., Proc. Natl. Acad. Sci. U.S.A., 1983, 80:4949-4953. Other methods of producing monoclonal antibodies, a hybridoma cell, or a hybridoma cell culture are also well known. See e.g., Antibodies: A Laboratory Manual, Harlow et al., Cold Spring Harbor Laboratory, 1988; or the method of isolating monoclonal antibodies from an immunological repertoise as described by Sasatry, et al., Proc. Natl. Acad. Sci. USA, 1989, 86:5728-5732; and Huse et al., Science, 1981, 246:1275-1281. The references cited are hereby incorporated herein by reference.

In one embodiment of the invention, monoclonal antibodies specific for PKC-zeta can be used as a delivery vehicle for drug or toxin. Drug or toxin can be conjugated to the antibodies using a biochemical approach. Monoclonal antibodies specific for the amino-terminus of PKC-zeta can be used as a delivery vehicle for drug or toxin. This enables the transport of drug or toxin to tumor cells with high expression of PKC-zeta.

Embodiments of peptides that inhibit PKC-zeta activity are described in Published International Patent Application W01993020101.

Embodiments of compound inhibitors of PKC-zeta are described in U.S. Patent Application Publication No. 2009/0318462.

In some embodiments, PKC-zeta inhibitors useful according to the present invention are agents that reduce or inhibit the expression of PKC-zeta, such as agents that inhibit the transcription, translation, and/or processing of PKC-zeta.

In an embodiment, the invention provides a method of screening for PKC-zeta inhibitors as useful candidates for treatment of breast tumorigenesis or breast cancer, comprising a PKC-zeta inhibitor; contacting tumorigenesis or cancerous breast cells with the PKC-zeta inhibitor, determining whether growth or proliferation of the tumorigenesis or cancerous breast cells is slowed; and, if so, identifying the PKC-zeta inhibitor as a useful candidate for treatment of breast tumorigenesis or breast cancer.

In an embodiment, the PKC-zeta inhibitor is a PKC-zeta antisense polynucleotide. In an embodiment, the PKC-zeta inhibitor is an antisense polynucleotide that targets human PKC-zeta mRNA. In some embodiments, the PKC-zeta antisense polynucleotides target PKC-zeta mRNAs of non-human animals including, but not limited to, apes, chimpanzees, orangutans, monkeys, dogs, cats, horses, pigs, sheep, goats, mice, rats, and guinea pigs. The skilled artisan would readily appreciate that the antisense polynucleotides can be designed to target any PKC-zeta mRNAs publicly known.

In some embodiments, the PKC-zeta inhibitor is a siRNA having a sequence sufficiently complementary to a target PKC-zeta mRNA sequence to direct target-specific RNA interference (RNAi). In some embodiments, the PKC-zeta inhibitor is siRNA having a sequence sufficiently complementary to a target human PKC-zeta mRNA sequence (such as mRNA encoding SEQ ID NO:1) to direct target-specific RNA interference.

Examples of antisense polynucleotides include, but are not limited to, single-stranded DNAs and RNAs that bind to complementary target PKC-zeta mRNA and inhibit translation and/or induce RNaseH-mediated degradation of the target transcript; siRNA oligonucleotides that target or mediate PKC-zeta mRNA degradation; ribozymes that cleave PKC-zeta mRNA transcripts; and nucleic acid aptamers and decoys, which are non-naturally occurring oligonucleotides that bind to and block PKC-zeta protein targets in a manner analogous to small molecule drugs.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The terms “nucleic acid” or “nucleic acid sequence” encompass an oligonucleotide, nucleotide, polynucleotide, or a fragment of any of these, DNA or RNA of genomic or synthetic origin, which may be single-stranded or double-stranded and may represent a sense or antisense strand, peptide nucleic acid (PNA), or any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog”, also referred to herein as an “altered nucleotide” or “modified nucleotide,” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of endogenous target genes, such as PKC-zeta.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

As used herein, a siRNA having a “sequence sufficiently complementary to a target mRNA sequence to direct target-specific RNA interference (RNAi)” means that the siRNA has a sequence sufficient to trigger the destruction of the target mRNA (e.g., PKC-zeta mRNA) by the RNAi machinery or process. “mRNA” or “messenger RNA” or “transcript” is single-stranded RNA that specifies the amino acid sequence of one or more polypeptides. This information is translated during protein synthesis when ribosomes bind to the mRNA.

The present invention also contemplates vectors (e.g., viral vectors) and expression constructs comprising the nucleic acid molecules useful for inhibiting PKC-zeta expression and/or activity. In an embodiment, the vector comprises a siRNA that targets PKC-zeta mRNA. In another embodiment, the vector comprises a nucleic acid molecule encoding an anti-PKC-zeta antibody.

As used herein, the term “expression construct” refers to a combination of nucleic acid sequences that provides for transcription of an operably linked nucleic acid sequence. As used herein, the term “operably linked” refers to a juxtaposition of the components described, wherein the components are in a relationship that permits them to function in their intended manner. In general, operably linked components are in contiguous relation.

Expression constructs of the invention will also generally include regulatory elements that are functional in the intended host cell in which the expression construct is to be expressed. Thus, a person of ordinary skill in the art can select regulatory elements for use in, for example, bacterial host cells, yeast host cells, mammalian host cells, and human host cells. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

### Therapeutic Compositions and Formulations

The present invention further provides therapeutic compositions that contain an effective amount of a therapeutic agent and a pharmaceutically acceptable carrier or adjuvant.

The therapeutic agent can be formulated in a variety of forms. These include for example, solid, semi-solid, and liquid dosage forms, such as tablets, pills, powders, liquid solutions or suspensions, suppositories, and injectable and infusible solutions. The preferred form depends on the intended mode of administration and therapeutic application.

In a preferred embodiment, the composition is formulated in accordance with routine procedures as a pharmaceutical composition adapted for local injection administration to human beings. Typically, compositions for local injection administration are solutions in sterile isotonic aqueous buffer. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile water for injection or saline can be provided so that the ingredients may be mixed prior to administration.

The present invention also provides for a therapeutic method by administering therapeutic or pharmaceutical compositions in a form that can be combined with a pharmaceutically acceptable carrier. In this context, the compound may be, for example, isolated or substantially pure. The term “carrier” refers to a diluent, adjuvant, excipient, or vehicle with which the compound is administered. Such pharmaceutical carriers can be sterile liquids, such as water and oils, including those of petroleum oil such as mineral oil; vegetable oil such as peanut oil, soybean oil, and sesame oil; animal oil; or oil of synthetic origin.

Suitable carriers also include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic composition, if desired, can also contain minor amounts of wetting or emulsifying agents, or pH buffering agents.

The amount of active ingredient that may be combined with the carrier materials to produce a single dosage form will vary, depending on the type of the condition and the subject to be treated. The amount of active ingredient that may be combined with the carrier materials to produce a single dosage form will vary, depending on the type of the condition and the subject to be treated. In general, a therapeutic composition contains from about 5% to about 95% active ingredient (w/w). More specifically, a therapeutic composition contains from about 20% (w/w) to about 80% or about 30% to about 70% active ingredient (w/w).

The therapeutic agents of the invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the present invention.

The therapeutic or pharmaceutical compositions of the present invention can also be formulated as neutral or salt forms. Pharmaceutically acceptable salts include, but are not limited to, hydrochloric, phosphoric, acetic, oxalic, sodium, potassium, ammonium, calcium, ferric hydroxides, isopropylamine, and triethylamine salts.

### Routes of Administration

The therapeutic agents and compositions of the present invention can be administered to the subject being treated by standard routes, including oral, or parenteral administration including intravenous, intramuscular, and intraspinal injection, infusion, and electroporation, as well as co-administration as a component of any medical device or object to be inserted (temporarily or permanently) into a subject.

In some embodiments, the methods disclosed herein include contacting a malignant breast tumor or malignant breast tumor cells with an effective amount of a PKC-zeta inhibitor. In some embodiments, the PKC-zeta inhibitor comprises a polynucleotide (including recombinant expression vectors encoding PKC-zeta antisense RNA, intracellular PKC-zeta antibodies).

The amount of the therapeutic or pharmaceutical composition of the present invention effective in the treatment of breast cancer and/or inhibition of breast tumorigenesis will depend on a variety of factors, such as the route of administration and the seriousness of the condition, and should be decided according to the judgment of the practitioner and each patient's circumstances. In general, the dosage ranges from about 0.01 μg/kg to about 10 mg/kg, about 0.01 μg/kg to about 1 mg/kg, about 0.01 μg/kg to about 100 μg/kg, about 0.01 μg/kg to about 10 μg/kg, or about 0.01 μg/kg to about 1 μg/kg. Such a unit dose may be administered once to several times (e.g. two, three and four times) every two weeks, every week, or every day.

In one embodiment, the therapeutic agents and compositions of the present invention and any second therapeutic agent are administered simultaneously or sequentially to the patient, with the second therapeutic agent being administered before, after, or both before and after treatment with the compounds of the present invention. Sequential administration may involve treatment with the second therapeutic agent on the same day (within 24 hours) of treatment with the subject compound. Sequential administration may also involve continued treatment with the second therapeutic agent on days that the subject compound is not administered.

Following is an example that illustrates embodiments for practicing the invention. The example should not be construed as limiting.

### Example 1

### Overexpression of PKC-Zeta in Malignant Breast Tumors

To investigate the effects of PKC-ζ on breast tumorigenesis, Western blots probing for PKC-ζ is performed on 2 normal breast tissue samples, 7 non-cancerous, benign breast tissue samples, and 12 malignant breast tumor samples. Statistical analysis is performed by a student's T-test. The level of PKC-ζ is considered as different if P<=0.01.

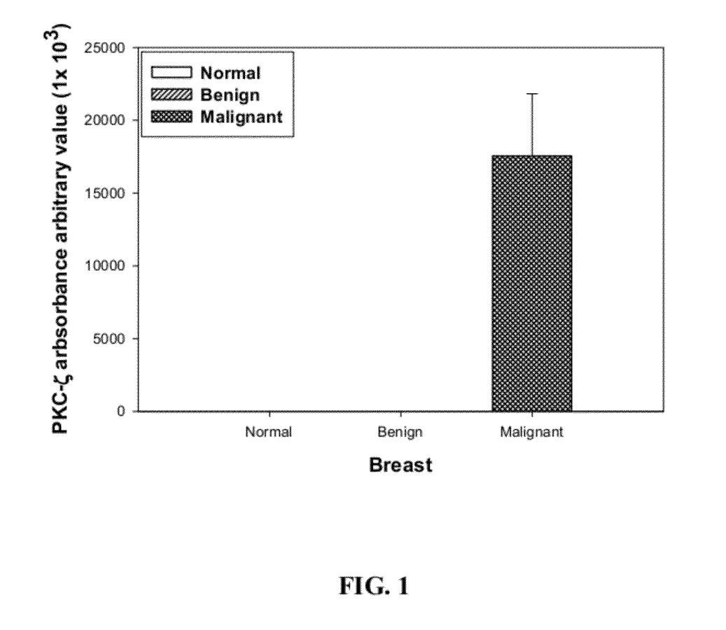

As shown in FIG. 1, almost no PKC-ζ is detected in normal breast or benign breast tissue. In contrast, PKC-ζ is robustly expressed in malignant breast tumor tissue. The increase in PKC-ζ in malignant breast cancer biopsies is 18,000 fold, when compared to PKC-ζ level in normal and benign breast tissue. Specifically, with respect to PKC-ζ level in normal/malignant tissue, the T value is 4.0959389049 and the P value is 0.0149024756; with respect to PKC-ζ level in benign/malignant tissue, the T value is 4.0959389049 and the P value is 0.0149024756.

These results show that PKC-ζ can be used as a biomarker for breast cancer tumorigenesis. In addition, reducing the level of, or inhibiting the expression and/or activity of PKC-ζ, can be used to prevent and/or treat breast cancer.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

What is claimed is:

1. A method for diagnosing the presence of breast tumorigenesis or breast cancer in a subject, comprising:
(a) obtaining a biological sample from a subject;
(b) detecting in the sample a level of expression for PKC-zeta; and
(c) comparing the expression level in (b) to a level of expression in a normal control, wherein overexpression of PKC-zeta, with respect to the control, indicates the presence of breast tumorigenesis or breast cancer in the subject.

2. The method according to claim 1, wherein the biological sample is obtained from a breast of the subject.

3. The method according to claim 1, wherein the subject is a human.

4. The method according to claim 1, wherein the comparing step includes: contacting the sample with an antibody that specifically recognizes PKC-zeta protein; and detecting the complex between the antibody and the PKC-zeta protein.

5. The method according to claim 4, wherein said PKC-zeta protein is contacted with an antibody that specifically recognizes PKC-zeta in an immunoassay selected from the group consisting of radioimmunoassay, western blot assay, immunofluorescent assay, enzyme immunoassay, immunoprecipitation, chemiluminescent assay, immunohistochemical assay, dot blot assay, and slot blot assay.

6. A method for predicting whether a subject is at risk of recurrence of breast tumorigenesis or breast cancer, comprising:
(a) obtaining a biological sample from a subject who has received treatment for breast cancer;
(b) detecting in the sample a level of expression for PKC-zeta; and
(c) comparing the expression level in (b) to a level of expression in a normal control, wherein overexpression of PKC-zeta, with respect to the control, indicates that the subject is at risk of recurrence of breast tumorigenesis or breast cancer.

7. The method according to claim 6, wherein the biological sample is obtained from a breast of the subject.

8. The method according to claim 6, wherein the comparing step includes: contacting the sample with an antibody that specifically recognizes PKC-zeta protein; and detecting the complex between the antibody and the PKC-zeta protein.

9. The method according to claim 6, wherein the subject had received treatment for breast cancer and, as a result of the treatment, does not have detectable breast cancer cells.

10. The method according to claim 9, wherein the biological sample is obtained from a breast of the subject.

11. A method of inhibiting breast tumorigenesis or breast cancer in a subject, comprising administering to a subject in need of such treatment an agent that inhibits PKC-zeta, whereby breast tumorigenesis is inhibited.

12. The method according to claim 11, wherein the agent is a PKC-zeta-specific small interfering RNA molecule.

13. The method according to claim 11, wherein the agent is a PKC-zeta-specific antibody.

14. The method according to claim 11, wherein the subject has breast cancer or breast tumorigenesis.

15. A method for screening for PKC-zeta inhibitors as useful candidates for treatment of breast tumorigenesis or breast cancer, comprising:
a PKC-zeta inhibitor;
contacting tumorigenesis or cancerous breast cells with the PKC-zeta inhibitor;
determining whether growth or proliferation of the tumorigenesis or cancerous breast cells is slowed; and, if so,
identifying the PKC-zeta inhibitor as a useful candidate for treatment of breast tumorigenesis or breast cancer.


SEQUENCE LISTING (1 sequence)

SEQ ID NO: 1 - protein, 592 aa, homosapien [sequence omitted]